(19) United States
(12) Patent Application Publication
(10) Pub. No.: US 2022/0218926 A1
(43) Pub. Date: Jul. 14, 2022

(54) INHALER AND METHOD OF DETECTING OBSTRUCTION

(71) Applicant: KINDEVA DRUG DELIVERY L.P., Woodbury, MN (US)

(72) Inventor: John A. ALIMI, Cambridge (GB)

(73) Assignee: KINDEVA DRUG DELIVERY L.P., Woodbury, MN (US)

(21) Appl. No.: 17/608,313

(22) PCT Filed: Apr. 30, 2020
(86) PCT No.: PCT/IB2020/054080

§ 371 Date: Nov. 2, 2021

Related U.S. Application Data
(60) Provisional application No. 62/844,556, filed on May 7, 2019.

(51) Int. Cl. A61M 15/00

(52) U.S. Cl. CPC A61M 15/009; A61M 15/0091; A61M 15/0021; A61M 2205/13; A61M 2205/18; A61M 2205/582; A61M 2205/3368; A61M 2205/332; A61M 2205/581; A61M 2205/583; A61M 2205/3306

(57) ABSTRACT

An inhaler for delivering a medicament to a patient includes an actuator housing for holding the medicament. The actuator housing includes an air inlet for receiving air flow. The actuator housing further defines an air flow path into which the medicament is dispensed. The inhaler also includes a sensor disposed proximal to the air inlet. The sensor is configured to detect an obstruction of the air inlet and to generate an output signal indicative of the obstruction. The inhaler further includes a feedback device configured to receive the output signal from the sensor and to generate a feedback signal. The feedback signal indicates an obstruction of the air inlet.

### TECHNICAL FIELD

The present disclosure generally relates to inhalation devices and, more particularly, to detecting an obstruction of an air inlet of inhalation devices and a method of detecting the obstruction.

### BACKGROUND

For various reasons, a certain percentage of patients suffering from chronic illnesses, such as asthma and chronic obstructive pulmonary disease (COPD), do not take their prescription as described. This can inhibit patient improvement and cause disease progression. Hence, adherence programs can be used to measure an extent to which patients follow their prescribed medication for treatment of their health condition.

Inhalers for pulmonary delivery, whether they be press-and-breathe or breath-actuated type devices, can deliver a medicament to an oral cavity of a patient. The medicament is delivered through an orifice which is in fluid communication with a fluid source, such as a canister.

Press-and-breathe and breath-actuated inhalers often include an air inlet that allows an inflow of air into the inhaler such that the medicament released from a reservoir or a canister enters this air flow. It is important not to obstruct the air inlet when administering the medicament. Obstruction of the air inlet whilst administering the medicament can reduce the operational effectiveness of an inhaler. A certain percentage of users knowingly or unknowingly obstruct the air inlet.

### SUMMARY

In one aspect, the present disclosure relates to an inhaler for delivering a medicament to a patient. The inhaler includes an actuator housing for holding the medicament. The actuator housing includes an air inlet for receiving air flow. The actuator housing further defines an air flow path into which the medicament is dispensed. The inhaler also includes a sensor. The sensor is configured to detect an obstruction of the air inlet and to generate an output signal indicative of the obstruction. The inhaler further includes a feedback device configured to receive the output signal from the sensor and to generate a feedback signal that indicates obstruction of the air inlet.

In another aspect, the present disclosure relates to a method of detecting an obstruction of an air inlet of an inhaler. The inhaler is used for delivering a medicament to a patient. The method includes detecting the obstruction of the air inlet by a sensor. The method includes generating an output signal indicative of the obstruction by the sensor. The method includes receiving the output signal from the sensor at a feedback device. The method includes generating a feedback signal by the feedback device indicating obstruction of the air inlet.

In another aspect, the present disclosure relates to an inhaler for delivering a medicament to a patient. The inhaler includes an actuator housing for holding the medicament. The actuator housing includes an air inlet for receiving air flow. The actuator housing further defines an air flow path into which the medicament is dispensed. The inhaler also includes an add-on device detachably connected to the actuator housing. The add-on device includes a sensor. The sensor is configured to detect an obstruction of the air inlet and to generate an output signal indicative of the obstruction. The add-on device also includes a feedback device configured to receive the output signal from the sensor and to generate a feedback signal. The feedback signal indicates obstruction of the air inlet.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a perspective view of an inhaler according to embodiments of the present disclosure;

FIG. 2 is a perspective view of an air inlet cover associated with the inhaler depicted in FIG. 1;

FIG. 3 is a schematic representation of the inhaler depicted in FIG. 1 having a sensor disposed on an actuator housing of the inhaler according to embodiments of the present disclosure;

FIG. 4 is a block diagram illustrating a system associated with the inhaler depicted in FIG. 1 for detecting an obstruction of an air inlet of the inhaler according to embodiments of the present disclosure;

FIG. 5 is a schematic representation of the inhaler depicted in FIG. 1 having the sensor disposed obliquely on the actuator housing of the inhaler according to embodiments of the present disclosure;

FIG. 6 is a schematic representation of an inhaler having the sensor disposed on an add-on device associated with the inhaler according to embodiments of the present disclosure;

FIG. 7 is a schematic representation of an inhaler having a sensor mounted at a bottom portion of the add-on device associated with the inhaler according to embodiments of the present disclosure;

FIG. 8 is a schematic representation of the inhaler depicted in FIG. 1 having the sensor mounted in an air flow path defined by the actuator housing according to embodiments of the present disclosure;

FIG. 9A is a schematic representation of an inhaler having a sensor disposed generally parallel to an air flow path defined by an actuator housing and a cover member of the inhaler in an open position according to embodiments of the present disclosure;

FIG. 9B is a schematic representation of the inhaler depicted in FIG. 9A illustrating the cover member of the inhaler in a closed position according to embodiments of the present disclosure;

FIG. 10A is a schematic representation of an inhaler having a sensor disposed generally parallel to an air flow path defined by an actuator housing and a cover member of the inhaler in an open position according to embodiments of the present disclosure;

FIG. 10B is a schematic representation of the inhaler depicted in FIG. 10A illustrating the cover member of the inhaler in a closed position according to embodiments of the present disclosure;

and

FIG. 11 is a flowchart for a method of detecting the obstruction of the air inlet of the inhaler.

### DETAILED DESCRIPTION

In the following description, reference is made to the accompanying figures that form a part thereof and in which various embodiments are depicted by way of illustration. It is to be understood that other embodiments are contemplated and may be made without departing from the scope or spirit of the present disclosure. The following detailed description, therefore, is not to be taken in a limiting sense.

The present disclosure will be described with respect to particular embodiments and with reference to certain drawings, but the disclosure is not limited thereto. The drawings described are only schematic and are non-limiting. In the drawings, the size of some of the elements may for illustrative purposes be exaggerated and not drawn to scale.

It will be understood that the terms “vertical”, “horizontal”, “top”, “bottom”, “above”, “below”, “left”, “right” etc. as used herein refer to particular orientations of the figures and these terms are not limitations to the specific embodiments described herein.

Common inhalers include pressurized metered-dose inhalers (pMDIs), dry powder inhalers (DPIs), and soft mist inhalers (SMIs), all of which have drug reservoirs and airflow paths extending from an air inlet to an air outlet. Examples of inhalers are described in International Patent Application Publications WO 2017/112400 and WO2015/034709, which are incorporated herein by reference in their entirety. Although subsequent description is for specific embodiments of pMDIs, the present disclosure can be applicable to all types of inhalers. In embodiments, a pMDI comprises a canister-retaining or tubular housing portion and a tubular mouthpiece portion, the tubular mouthpiece portion being angled with respect to the tubular housing portion. An air inlet is defined at an upper end and/or a lower end of the tubular housing portion. Proximal to the lower end of the tubular housing portion, a thumb grip is provided. Further, a metering valve is disposed within the tubular housing portion that releases a metered amount of medicament from a canister or reservoir of the inhaler. In operation of the inhaler, a plume of medicament produced from an orifice that is in communication with the metering valve is introduced into the tubular mouthpiece portion and is inhaled by a patient through the tubular mouthpiece portion. However, as described above, there can sometimes be an undesirable obstruction at the air inlet of the inhaler.

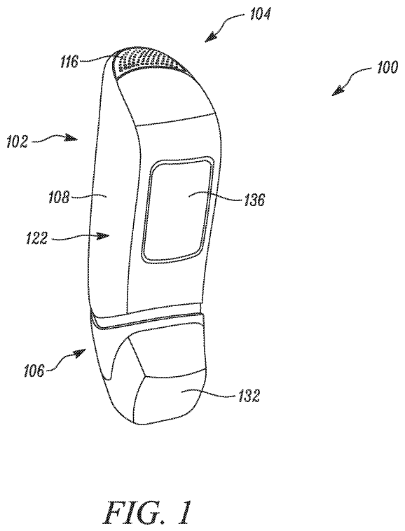

FIG. 1 illustrates a perspective view of an inhaler 100 for delivering a medicament to a patient. The inhaler 100 may be embodied as an electronic inhaler. Further, the inhaler 100 may include an onboard power source (not depicted), such as batteries or cells, that powers various electronic components of the inhaler 100 (including the sensors, receivers, and feedback devices described below). In an embodiment, the inhaler 100 is a press-and-breathe inhaler. Such a press-and-breathe inhaler includes a housing and a mouthpiece defined at a lower section of the housing. Further, the housing receives a canister having a generally cylindrical structure and a metering valve. The canister releases a spray of medicament when the canister is depressed by the patient. In such inhalers, the spray may be introduced directly into the patient's mouth, nasal area, or respiratory airways. Such devices can be actuated by pressure applied by the patient's fingers, button action, or other related manual techniques.

In another embodiment, the inhaler 100 is a breath-actuated inhaler. In such a case, the metering valve may be actuated by a pressure differential created by inhalation of a patient to automatically dispense a spray of the medicament without any manual intervention. In some embodiments, the inhaler 100 may have to be primed by the patient before breath-actuated inhalation. The inhaler 100 may be primed by moving a priming actuator, such as a lever or a mouthpiece cover. The inhaler 100 includes an actuator housing 102 for holding the medicament. The actuator housing 102 defines an air flow path “F” into which the medicament is dispensed. The air flow path “F” may be defined as a flow path that an air flow follows while flowing through the inhaler 100. The actuator housing 102 includes a housing portion 108. The housing portion 108 has a substantially hollow structure and is tubular in shape.

A canister (not depicted) is removably received within the housing portion 108. The canister contains a fluid formulated with the medicament and is embodied as an aerosol canister. In another embodiment, the fluid formulated with the medicament may be stored in a reservoir. The canister may have a generally cylindrical structure. The canister includes a metering valve (not depicted) for metering an amount of the medicament exiting the canister corresponding to a single spray pattern or spray plume. The canister releases a predetermined amount of the medicament through the metering valve upon actuation. The canister further includes a valve stem (not depicted) extending from the metering valve. At a closed bottom end of the actuator housing 102 sits a nozzle block (not depicted) that includes a stem socket (not depicted). The stem socket is provided for receiving the valve stem of the canister. The stem socket includes an exit orifice (not depicted) or actuator nozzle (not depicted) communicating with the mouthpiece 106 of the inhaler 100.

The actuator housing 102 defines an outer surface 122. The outer surface 122 has a grip section (not depicted). The grip section is can be proximate to the bottom end of the actuator housing 102. The grip section allows a user to hold the inhaler 100 during use. The grip section may optionally include a set of protrusions, a set of indents, or a sleeve to provide an enhanced gripping surface. The actuator housing 102 can optionally include a display device 136 for providing notifications to the patient. For example, the display device 136 may notify the patient when the medicament in the canister is about to deplete.

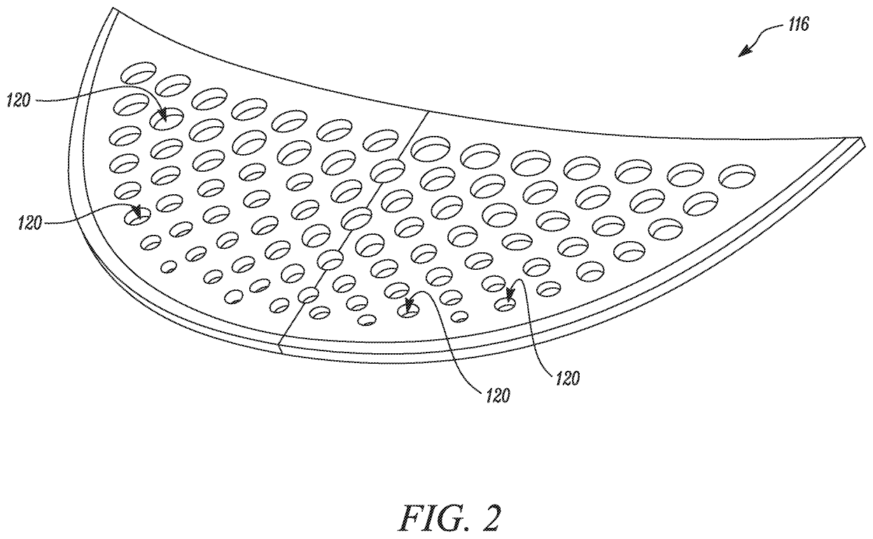

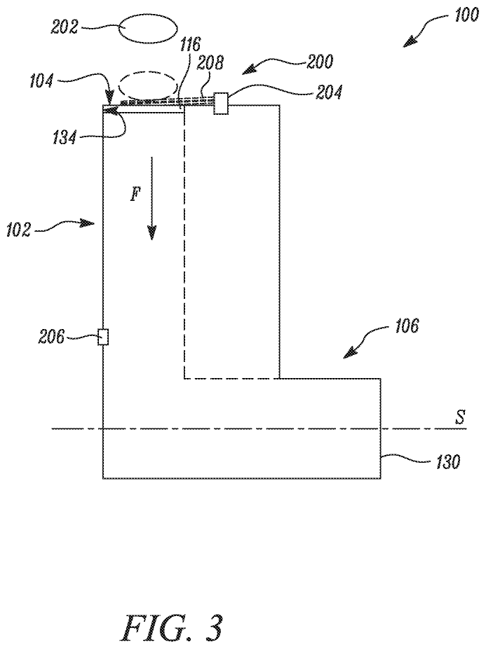

Further, the actuator housing 102 includes an air inlet 104 for receiving the air flow. The air inlet 104 is defined at an upper end of the actuator housing 102. In alternative embodiments, the air inlet 104 is defined at a lower end of the actuator housing 102. The actuator housing 102 can include an air inlet cover 116 that at least partially outlines one or more openings of the air inlet 104. The air inlet cover 116 may be embodied as a grille. As depicted in the illustrated embodiment of FIG. 2, the air inlet cover 116 can have several apertures 120 that allow the air flow to enter the inhaler 100. The air inlet cover 116 is generally semi-circular in shape in the illustrated embodiment. It should be understood, however, that the air inlet cover is not limited to this shape and can be other shapes in other embodiments. In the illustrated embodiment, the apertures 120 have a generally circular shape. However, in other embodiments, the apertures 120 may have any other non-circular shape, for example, polygonal, elliptical, rectangular and so forth. In various examples, a diameter of the apertures 120 may vary across the air inlet cover 116. Further, the actuator housing 102 also defines a wall 134 (depicted in FIG. 3), such that in the illustrated embodiment the air inlet 104 is defined by the wall 134 and the air inlet cover 116. In other embodiments, the air inlet 104 is defined by the wall 134 and the air inlet cover 116 is optional.

The air inlet can be an opening in the actuator housing of any shape or size that provides for the required air flow to the device and is not necessarily limited to any specific position in the housing. The air inlet can be a simple uncovered opening in the housing or it can contain a cover.

In embodiments with an air inlet cover, the air inlet cover at least partially covers the air inlet. The air inlet cover can help to direct airflow and to partially limit the ingress of contaminants such as dust, dirt, liquids, and other environmental debris into the internal portion of the inhaler. The cover portion of the air inlet can have openings to allow for the inflow of air. For example, the air inlet cover may be in the form of a screen, grille, vents, a plate with one or more apertures, or a plate with elongated slots. The air inlet cover may be a separate part affixed to the actuator or may be integrated into the housing. For example, the housing can be made as a molded plastic part and the cover of the air inlet (such as a screen or grille) is incorporated as part of the molded housing. Referring to FIG. 1, the mouthpiece is embodied as a generally tubular portion extending from the actuator housing 102. The mouthpiece 106 is joined to the housing portion 108. In an example, the mouthpiece 106 is angled with respect to the actuator housing 102. The mouthpiece 106 may have a circular cross-section or a non-circular cross-section, such as an elliptical or oblong cross-section. Further, the mouthpiece 106 has a substantially hollow structure.

The mouthpiece 106 defines a mouthpiece end 130 (depicted FIG. 3). A user or the patient may put at least a part of the mouthpiece end 130 into his mouth for using the inhaler 100. The mouthpiece end 130 may include a cross-section perpendicular to an axis of flow “S”. The axis of flow “S” may be defined a general direction in which a spray of metered medicament is dispensed through the mouthpiece 106. The shape of the mouthpiece end 130 may include any suitable shape such as circular, a substantially oval shape, a substantially elliptical shape, or a polygonal shape. The present disclosure is not limited by the shape of the cross-section of the mouthpiece end 130.

The mouthpiece 106 may be provided with a dust cap 132. In one example, the dust cap 132 is pivotable between a closed position and an open position. In use, the patient may rotate the dust cap 132 to its open position and insert the exposed mouthpiece 106 into their mouth. Alternatively, the dust cap 132 can be detached from the inhaler to use the inhaler 100 and may be reattached after using the inhaler 100.

When the inhaler 100 is a breath actuated inhaler design, inhalation by the patient through the mouthpiece 106, produces a pressure differential in the actuator housing 102 that causes the breath-actuated mechanism to automatically displace the canister relative to the valve stem. The medicament contained within the metering chamber of the canister is accordingly released in response to the patient's inspiration.

During the patient's inspiration, air flows from the air inlet 104, through the actuator housing 102, to the mouthpiece 106, and then to the patient. The medicament released from the metering chamber of the canister enters this air flow. Thus, during operation of the inhaler 100, a plume of the medicament produced from the exit orifice or the actuator nozzle is inhaled by the patient through the mouthpiece 106. After inhalation of a dose of the medicament by the patient, the dust cap 132 can be returned to its closed position.

In some cases during inspiration, the air inlet may be at least partially blocked by an object such as a finger or a thumb of the patient which in turn blocks the air flow entering through the air inlet 104. Such blockage of the air inlet 104 may lead to a malfunction of the breath-actuated mechanism and/or cause incomplete or ineffective delivery of the medicament to the patient.

In some cases, during inspiration the air inlet cover 116 may be at least partially blocked by an object such as a finger or a thumb of the patient which in turn blocks the air flow entering through the air inlet 104. Such blockage of the air inlet 104 may lead to a malfunction of the breath-actuated mechanism and/or cause incomplete or ineffective delivery of the medicament to the patient.

In some cases, during inspiration at least one opening in the air inlet cover 116 may be blocked by an object such as a finger or a thumb of the patient which in turn blocks the air flow entering through the air inlet 104. Such blockage of the air inlet 104 may lead to a malfunction of the breath-actuated mechanism and/or cause incomplete or ineffective delivery of the medicament to the patient.

Referring to FIG. 3, the present disclosure is directed towards a system 200 for detecting an obstruction of the air inlet 104 of the inhaler 100. The obstruction may be caused by an object 202. e.g., one or more fingers or a thumb of the patient using the inhaler. The system 200 includes a sensor 204. The sensor 204 is disposed proximal to the air inlet 104. The sensor 204 is configured to detect the obstruction of the air inlet 104 and generate an output signal indicative of the obstruction. Further, the sensor 204 may be configured to emit detection signals 208 towards the air inlet 104. In an example, the sensor 204 may be configured to emit pulsed signals to detect the obstruction of the air inlet 104.

In embodiments, the sensor 204 can be activated based upon a detection of a predefined inhaler preparation signature and later deactivated after an inhalation maneuver has been completed. This may be desirable in order, for instance, to minimize power consumption in contrast to if the sensor was always active. It should be understood, however, that the sensor could be continuously active and the feedback device configured to distinguish between detected obstructions when the device is not being used and detected obstructions when the device is in use. In another embodiment, the sensor can be activated and deactivated using an on-off switch. It should be noted that the term “predefined inhaler preparation signature” used herein may refer to a parameter that indicates usage of the inhaler 100, such as, temperature, motion, orientation, and the like. Such parameters that correspond to the predefined inhaler preparation signature may be detected by sensors that may be present onboard the inhaler 100. For example, the sensor 204 may be activated based on a movement of the inhaler 100 detected by an accelerometer or any other motion sensor associated with the inhaler 100.

In some embodiments the sensor 204 is a proximity sensor.

Exemplary sensors 204 include an ultrasonic sensor, an optical sensor, a laser detector, a force sensor, and a temperature sensor. When the sensor 204 is an ultrasonic sensor, the ultrasonic sensor constantly pulses out wave signals and calculates nearness of a surface based on a time duration it takes to receive a return signal from the surface. Thus, detection of the object 202 within a range of the ultrasonic sensor results in generation of an output signal corresponding to the obstruction of the air inlet 104. A single ultrasonic element can be used for both emission and reception of the ultrasonic waves. A single oscillator can emit and receive ultrasonic waves alternately. Further, in an example, the ultrasonic sensor may be programmed to detect any obstruction within a predefined periphery of the air inlet 104 or the air inlet cover 116.

When the sensor 204 is an optical sensor, the optical sensor may include a sender and a receiver. The sender transmits detection signals which may be visible light signals or infrared light signals. The receiver constantly detects signals that reflect from a background environment, such as for example the wall 134 or the air inlet cover 116 or a separate reflector element. In such cases, the wall 134 of the air inlet 104, the air inlet cover 116, or the separate reflector element is designed to have a characteristic optical reflection. When the object 202 with a different surface characteristic, e.g., color, roughness, etc., is moved across a signal pathway, light reflection off the object 202 is detected to be different than light reflection from the background environment. This difference in light reflection is detected as an obstruction of the air inlet 104 and results in the generation of an output signal. The sender and receiver can be integrated together in the same sub-housing or they can be in separate housings.

In embodiments, the sensor can be configured as a through beam sensor. In a through beam sensor configuration the sender and receiver are oriented such that the light sending and light receiving elements are facing each other across a linear distance. The light signal can be transmitted directly to the receiver and the sensor operates by detecting whether an interruption in the signal has occurred. In operation, an interruption in the visible or infrared light signal by the object 202 may be detected as an obstruction of the air inlet 104 and results in the generation of an output signal.

When the sensor 204 is embodied as a laser detector, the laser detector includes a sender and a receiver. The sender constantly transmits detection signals and the receiver detects signals that are reflected by a background environment, such as the wall 134 or the air inlet cover 116. Changes in the laser signal may be analyzed to detect any obstructions.

Further, the sensor 204 may include force sensors, such as piezoelectric sensors, strain gauges, or Microelectromechanical systems (MEMs) based sensing technologies for measuring strain and capacitance. The sensor 204, embodied as a force sensor, may be positioned on or the air inlet cover 116, within the air inlet cover 116, or in contact with the air inlet cover 116. When a force exerted on the air inlet cover 116 exceeds a predefined threshold, an output signal is generated by the force sensor. Alternatively, the force sensor may be located adjacent to perimeter of the air inlet opening. When a force exerted on the force sensor adjacent to the air inlet opening exceeds a predefined threshold, an output signal is generated by the force sensor. The predefined threshold may in some embodiments be equal to zero or about zero as the air inlet 104 is ideally free of any obstructions. The force sensor may also be configured to detect a spike in force as the obstruction approaches the air inlet 104. The term force sensor includes touch sensors. In some embodiments, the force sensor is a resistive-type touch sensor.

In some embodiments, the force sensor is configured as a multi-layer laminate attached to air inlet cover and/or the housing adjacent to the air inlet opening. In some embodiments, the force sensor is configured as a multi-layer laminate incorporated within the air inlet cover and/or the housing adjacent to the air inlet opening.

Further, the sensor 204 may embody a temperature sensor that may be on the air inlet cover 116 or may be in contact with the air inlet cover 116. The temperature sensor calibrates to the room temperature upon activation of the inhaler 100 based on the predefined inhaler preparation signature. When the predefined inhaler preparation signature is embodied as temperature values, any change in temperature beyond a threshold temperature value for the predefined inhaler preparation signature results in generation of an output signal. Alternatively, the temperature sensor may be placed adjacent to perimeter of the air inlet opening. The temperature sensor may also be configured to detect a spike in temperature as the obstruction approaches the air inlet 104.

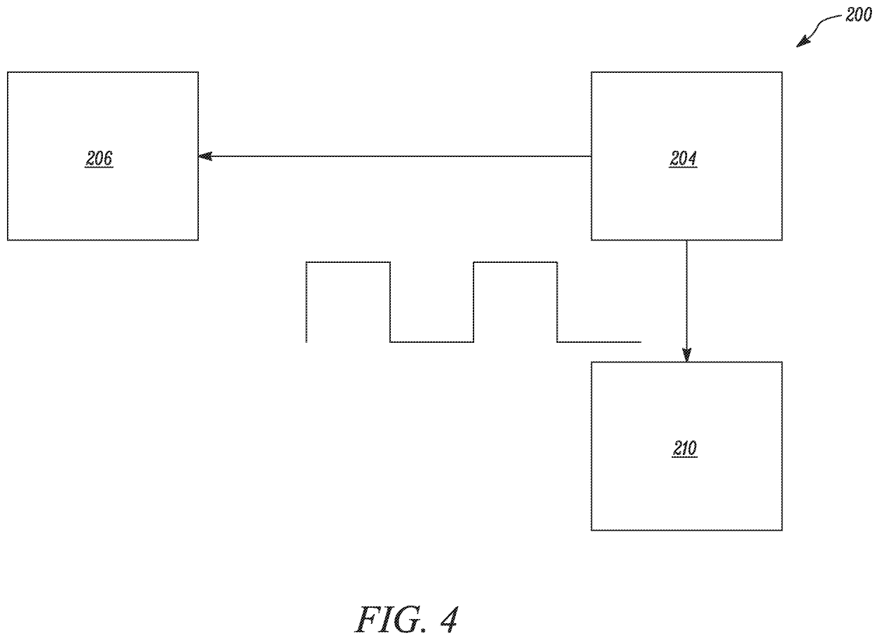

In the illustrated embodiment, the sensor 204 is disposed on the actuator housing 102. The sensor 204 is disposed generally perpendicular to the air flow path “F”. The sensor 204 is disposed proximal to the air inlet 104. More particularly, the sensor 204 is mounted on the air inlet cover 116. For example, the sensor 204 may be mounted on an upper surface of the air inlet cover 116. A sensitivity or range of the sensor 204 is programmed to be restricted to the periphery of the air inlet 104 and/or the air inlet cover 116, such that the sensor 204 can only detect obstructions caused by the object 202 around the periphery of the air inlet 104. When the sensor is activated, the sensor 204 continuously transmits detection signals. Further, a receiver 210 (depicted in FIG. 4) may be mounted on another end, for example, behind the air inlet 104, such that the receiver 210 receives signals reflected from the air inlet 104. If the object 202 comes within the range of the sensor 204, the sensor 204 detects the object 202 as the obstruction of the air inlet 104 and generates the output signal. It should be noted that the output signals may be sent to both adherence monitoring healthcare professionals and the patients.

Further, the system 200 includes a feedback device 206. In the illustrated embodiment, the feedback device 206 is disposed on the actuator housing 102. In other embodiments, the feedback device may be a separate component, such as a smartphone. The feedback device 206 may include one or more of an optical component, an audio component, and a haptic component. In one example, the display device 136 is embodied as the feedback device 206. As depicted in FIG. 4, the feedback device 206 is communicably coupled with the sensor 204. The feedback device 206 is configured to receive the output signal from the sensor 204 and generate a feedback signal indicative of the obstruction of the air inlet 104. It should be noted that the sensor 204 or the feedback device 206 may include control circuitry (not depicted) that processes signals generated by the sensor 204. The control circuitry may embody a single microprocessor or multiple microprocessors for receiving signals from various components of the system 200. Numerous commercially available microprocessors may be configured to perform the functions of the control circuitry. The control circuitry may further include a memory to store data and algorithms therein required for operation of the inhaler 100.

Further, the feedback device 206 notifies the patient regarding the obstruction of the air inlet 104 based on the receipt of the output signal. Thus, the feedback signal draws the patient's attention and hence prompts the patient to remove the finger or any other object that is obstructing the air inlet 104 (see FIGS. 1 and 3). The feedback device 206 may provide the feedback signal to the patient regarding the obstruction of the air inlet 104 using various methods, for example, a Light Emitting Diode (LED) which may display or flash a color signature to notify the patient regarding the obstruction of the air inlet 104, a buzzer sound that emits a sound frequency or tones to alert the patient, a haptic feedback, a text notification that may be displayed on the display device 136 (see FIG. 1) of the inhaler 100, an audible spoken word message, etc. The feedback device may also provide a feedback signal by wireless communication to a phone app.

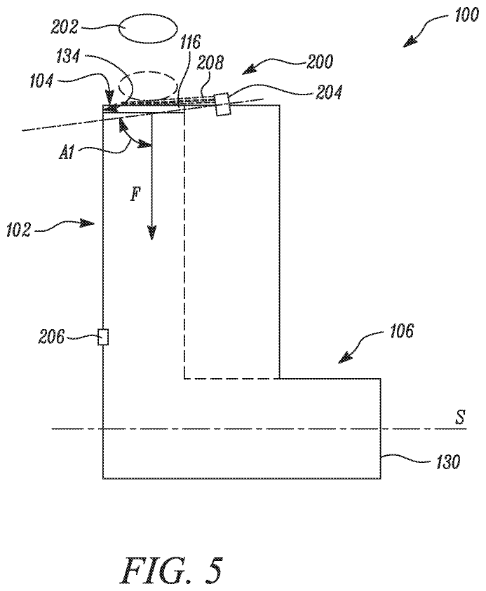

FIG. 5 illustrates another embodiment of the present disclosure. In this embodiment, the sensor 204 is angularly mounted. More particularly, the sensor 204 is disposed obliquely with respect to the air flow path “F”. In an example, an angle “μl” is defined between the sensor 204 and the air flow path “F”. In some embodiments, the angle “μl” is between about 10 degrees and about 85 degrees. In the illustrated embodiment, the sensor 204 is disposed proximal to the air inlet 104. In some embodiments, the sensor 204 may be attached to the air inlet cover or to the housing surrounding the air inlet. For example, the sensor 204 may be mounted on the upper surface of the air inlet cover 116.

When the sensor is activated, the sensor 204 continuously transmits the detection signals 208. Further, any obstruction caused by the presence of the object 202 across a pathway of the transmitted detection signals 208 is detected and the output signal is generated by the sensor 204. More particularly, miniature reflectors may be mounted on the wall 134 or the air inlet cover 116 such that the reflectors reflect the detection signals 208 transmitted from the sensor 204. If the object 202 comes within the range of the sensor 204, the sensor 204 detects the object 202 as the obstruction of the air inlet 104 and generates the output signal. The feedback device 206 receives the output signal from the sensor 204 and generates the feedback signal indicative of the obstruction of the air inlet 104. The feedback device 206 accordingly provides the feedback signal to the patient regarding the obstruction of the air inlet 104.

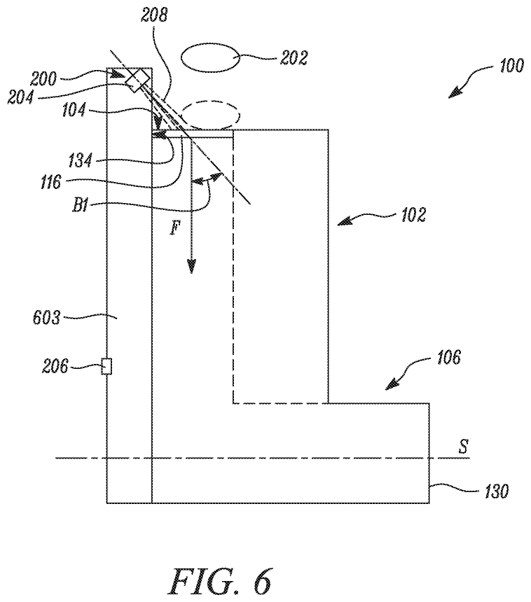

Referring now to FIG. 6, another embodiment of the present disclosure is illustrated. In this embodiment, the inhaler 100 includes an add-on device 603 for sensing obstruction of an air inlet. In some embodiments, the add-on device 603 can be an electronic adherence monitoring add-on device that monitors usage of the inhaler 100. The add-on device 603 is detachably connected to the actuator housing 102. In one example, the add-on device 603 may be connected to the actuator housing 102 by a snap-fit connection or a threaded joint. Alternatively, a tongue and groove joint, straps, hook and loop fasteners, and the like may be used to connect the add-on device 603 with the actuator housing 102. Further, the add-on device 603 may include a power source, such as batteries or cells, that may be present onboard the add-on device 603.

In this embodiment, the add-on device 603 includes the sensor 204 disposed proximal to the air inlet 104. More particularly, the sensor 204 is disposed on the add-on device 603. The sensor 204 may be embedded in the add-on device 603 such that it is near the air inlet 104. As illustrated, the sensor 204 is angularly mounted to the add-on device 603. More particularly, the sensor 204 is disposed obliquely with respect to the air flow path “F”. In an example, an angle “B 1” is defined between the sensor 204 and the air flow path “F”. In some embodiments, the angle “B 1” is between about 10 degrees and about 85 degrees. Further, the sensor 204 is programmed such that it is constantly sending detection signals towards the wall 134 of the air inlet 104 and/or the air inlet cover 116. Alternatively, the sensor 204 may be disposed generally perpendicular to the air flow path “F”. In such an example, the range of the sensor 204 may be programmed to be restricted to the periphery of the air inlet 104 and/or the air inlet cover 116, such that the sensor 204 can only detect obstructions caused by the object 202 around the periphery of the air inlet 104.

When the sensor is activated, the sensor 204 continuously transmits the detection signals 208. Further, any obstruction caused by the presence of the object 202 across the pathway of the transmitted detection signals 208 are detected and the output signal is generated by the sensor 204. More particularly, miniature reflectors may be mounted on the air inlet cover 116 such that the reflectors reflect the detection signals 208 transmitted from the sensor 204. If the object 202 comes within the range of the sensor 204, the sensor 204 detects the object 202 as the obstruction of the air inlet 104 and generates the output signal. The feedback device 206 receives the output signal from the sensor 204 and generates the feedback signal indicative of the obstruction of the air inlet 104. In this embodiment, the feedback device 206 is disposed on the add-on device 603. The feedback device 206 accordingly provides the feedback signal to the patient regarding the obstruction of the air inlet 104.

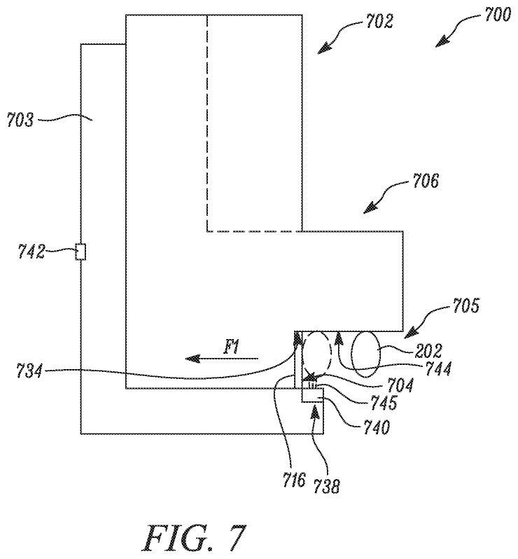

FIG. 7 illustrates another embodiment wherein an inhaler 700 includes an add-on device 703. The add-on device 703 may be detachably connected to the inhaler 700 by a snap-fit connection or a threaded joint. Alternatively, a tongue and groove joint, straps, hook and loop fasteners, and the like may be used to connect the add-on device 703 with an actuator housing 702. Further, the add-on device 703 may include a power source, such as batteries or cells, that may be present onboard the add-on device 703.

The add-on device 703 is similar in operation to the add-on device 603 depicted in FIG. 6. The inhaler 700 includes the actuator housing 702 and a mouthpiece 706. A function and structure of the actuator housing 702 and the mouthpiece 706 are similar to that of the actuator housing 102 and the mouthpiece 106, respectively, associated with the inhaler 100 depicted in FIG. 3. In this embodiment, the air inlet 704 is defined at a lower portion 705 of the inhaler 700. The air inlet 704 includes an air inlet cover 716 similar to the air inlet cover 116 depicted in FIG. 2. The inhaler 700 includes a system 738 that is similar to the system 200 described above. Further, the system 738 includes the sensor 740 and the feedback device 742 that are similar in operation to the sensor 204 and the feedback device 206, respectively, explained above. In this embodiment, the add-on device 703 includes the sensor 740 disposed proximal to the air inlet 704. The sensor 740 is disposed generally perpendicular to an air flow path “F1” defined by the actuator housing 702. In one example, a range of the sensor 740 is programmed to be restricted to a periphery of the air inlet 704 and/or the air inlet cover 716, such that the sensor 740 can only detect obstructions around the periphery of the air inlet 704.

When the sensor is activated, the sensor 740 continuously transmits detection signals 745. Further, any obstruction across a pathway of the detection signals 745 are detected and an output signal is generated by the sensor 740. More particularly, miniature reflectors may be mounted on the air inlet cover 716, such that the reflectors reflect the detection signals 745 transmitted from the sensor 740. If the object 202 comes within the range of the sensor 740, the sensor 740 detects the object 202 as the obstruction of the air inlet 704 and generates the output signal.

The feedback device 742 receives the output signal from the sensor 740 and generates a feedback signal indicative of the obstruction of the air inlet 704. In this embodiment, the feedback device 742 is disposed on the add-on device 703. Further, the feedback device 742 provides the feedback signal to the patient regarding the obstruction of the air inlet 704 based on the receipt of the output signal. For example, the feedback device 742 may provide the feedback signal to the patient regarding the obstruction of the air inlet 704 using an LED which may display or flash a color signature to notify the patient regarding the obstruction of the air inlet 704, a buzzer sound that emits a sound frequency or tones to alert the patient, a haptic feedback, a text notification that may be displayed on a display device (not depicted) of the inhaler 700, etc.

In another example, the sensor 740 may be programmed in such a way that it is constantly receiving signals returned from a bottom portion 744 of the mouthpiece 706. In yet another example, the sensor 740 is angularly mounted to the add-on device 703. More particularly, the sensor 740 is disposed obliquely with respect to the air flow path “F1”. In an example, an angle “Cl” is defined between the sensor 740 and the air flow path “F”. In some embodiments, the angle “Cl” is between about 10 degrees and about 85 degrees. Accordingly, the sensor 740 is programmed such that it is constantly receiving signals returned from the wall 734 of the air inlet 704 and/or the air inlet cover 716. Such an orientation of the sensor 740 can be used when the patients cover the air inlet 704 with their lips which is a common problem with inhalers having the air inlet 704 proximal to the mouthpiece 706.

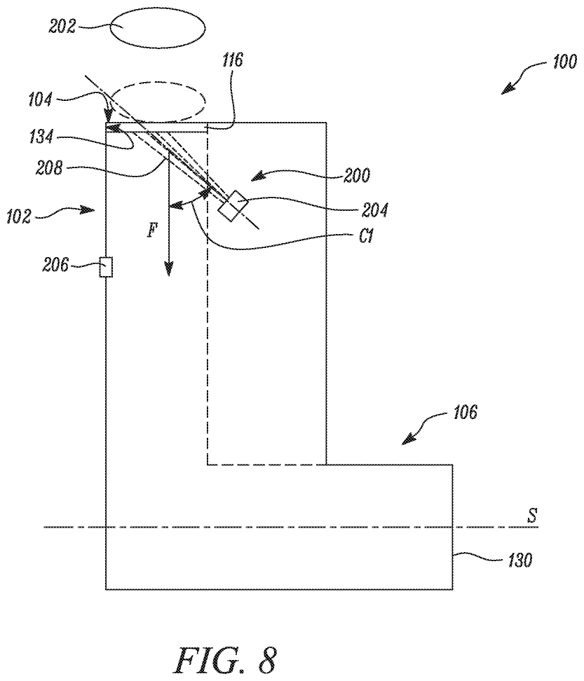

FIG. 8 illustrates the inhaler 100 according to another embodiment of the present disclosure. In this embodiment, the sensor 204 is mounted within the air flow path “F”. The sensor 204 is disposed proximal to the air flow path “F”. The sensor 204 is disposed obliquely to the air flow path “F”. Further, the sensor 204 is programmed such that it is constantly receiving signals returned from the wall 134 of the air inlet 104 and/or the air inlet cover 116. Alternatively, the sensor 204 is disposed generally perpendicular to the air flow path “F”. In such an example, the range of the sensor 204 may be programmed to be restricted to the periphery of the air inlet 104 and/or the air inlet cover 116 such that the sensor 204 can only detect obstructions caused by the object 202 around the periphery of the air inlet 104.

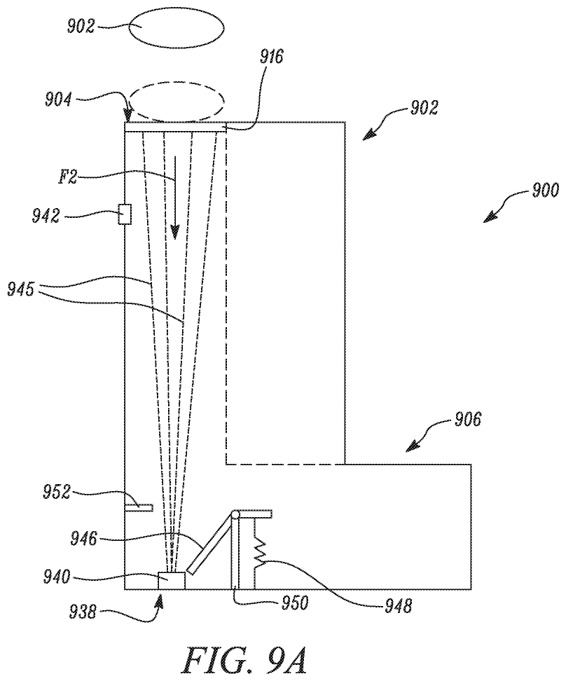

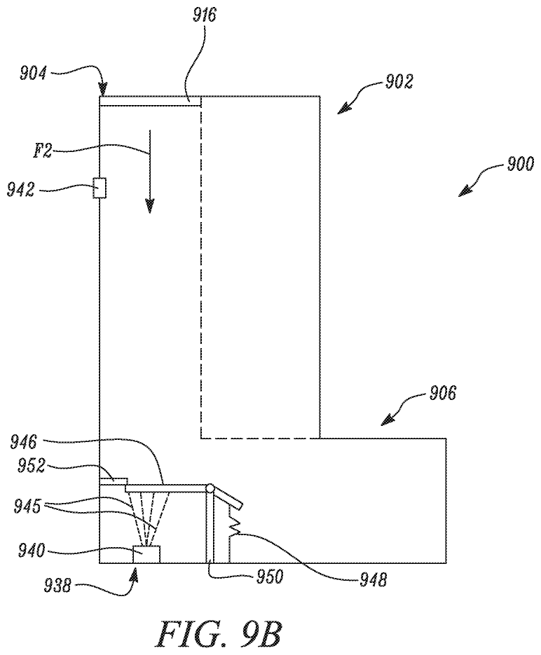

When the sensor is activated, the sensor 204 continuously transmits the detection signals 208. Further, any obstruction across the pathway of the detection signals 208 are detected and the output signal is generated by the sensor 204. More particularly, miniature reflectors may be mounted on the air inlet cover 116 such that they reflect the detection signals 208 transmitted from the sensor 204. In some examples, a reflected signal that varies from an ideal reflected signal may be registered as an obstruction in the signal pathway. If the object 202 comes within the range of the sensor 204, the sensor 204 detects the object 202 as the obstruction of the air inlet 104 and generates the output signal. The feedback device 206 receives the output signal from the sensor 204 and generates the feedback signal indicative of the obstruction of the air inlet 104. The feedback device 206 accordingly provides the feedback signal to the patient regarding the obstruction of the air inlet 104. Referring now to FIG. 9A, another embodiment of an inhaler 900 is illustrated. The inhaler 900 includes an actuator housing 902 and a mouthpiece 906. A function and structure of the actuator housing 902 and the mouthpiece 906 are similar to that of the actuator housing 102 and the mouthpiece 106, respectively, associated with the inhaler 100 depicted in FIG. 3. Further, the actuator housing 902 includes an air inlet 904. The air inlet 904 may include an air inlet cover 916 similar to the air inlet cover 116 depicted in FIG. 2. The inhaler 900 includes a system 938 that is similar to the system 200 described above. Further, the system 938 includes the sensor 940 and the feedback device 942 that are similar in operation to the sensor 940 and the feedback device 942, respectively, explained above. The sensor 940 is disposed generally parallel to an air flow path “F2” defined by the actuator housing 902. In an example, the range of the sensor 940 may be programmed to be restricted to a predefined range above a periphery of the air inlet 904 and/or the air inlet cover 916 such that the sensor 940 can only detect obstructions up to the predefined range above the periphery of the air inlet cover 916.

When the sensor is activated, the sensor 940 continuously transmits detection signals 945. The sensor 940 is programmed such that the air inlet cover 916 constantly reflects the detection signals 945 transmitted from the sensor 940. Any obstruction of the air inlet 904 alters the reflected signals that are detected by the sensor 940. If the signal alteration caused by the obstruction exceeds a predefined signal change, an output signal is generated by the sensor 940. The term “predefined signal change” may be defined as an allowable signal alteration which when exists does not compromise an effectiveness of the inhaler 900. The predefined signal change may be prestored in a memory of a control circuitry that may be associated with the sensor 940 or the feedback device 942. Thus, if the object 202 comes within the range of the sensor 940, the sensor 940 detects the object 202 as the obstruction of the air inlet 904 and generates the output signal.

The feedback device 942 receives the output signal from the sensor 940 and generates a feedback signal indicative of the obstruction of the air inlet 904. Further, the feedback device 942 provides the feedback signal to the patient regarding the obstruction of the air inlet 904 based on the receipt of the output signal. For example, the feedback device 942 may provide the feedback signal to the patient regarding the obstruction of the air inlet 904 using an LED which may display or flash a color signature to notify the patient regarding the obstruction of the air inlet 904, a buzzer sound that emits a sound frequency or tones to alert the patient, a haptic feedback, a text notification that may be displayed on a display device (not depicted) of the inhaler 900, etc.

The inhaler 900 also includes a cover member 946. The cover member 946 is movable between an open position and a closed position. The cover member 946 is depicted in the open position in FIG. 9A and in the closed position in FIG. 9B. In one example, the cover member 946 moves between the open and closed positions based on control signals received from a control module (not depicted) associated with the inhaler 100. More particularly, an actuator (not depicted) may move the cover member 946 based on the control signals received from the control module. Further, a spring 948 biases the cover member 946 to the open position, such that the actuator moves the cover member 946 against a biasing of the spring 948. The cover member 946 is pivotally coupled to a first member 950 such that the cover member 946 pivots with respect to the first member 950 to switch between the open and closed positions.

As depicted in FIG. 9B, the cover member 946 is configured to cover the sensor 940 in the closed position. More particularly, during the delivery of the medicament to the patient, the cover member 946 covers the sensors 940. In the closed position, the cover member 946 is in contact with a second member 952. The cover member 946 may include a flap, a vane, or a valve. The cover member 946 moves between the open and closed positions to isolate the sensor 940 from the air flow path “F2” during the delivery of the medicament to the patient. In another example, the sensor 940 may be further protected by a sensor shield such that the sensor shield does not affect a performance of the sensor 940. The cover member or sensor shield can be used to protect the sensor from possible contamination such as dust, dirt, or water.

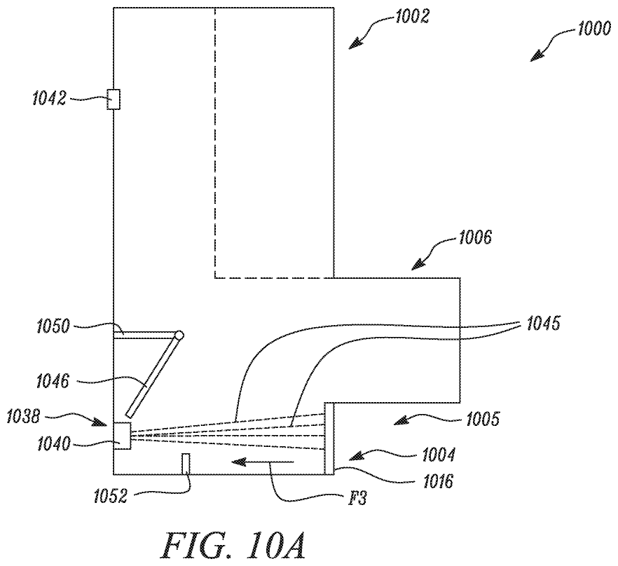

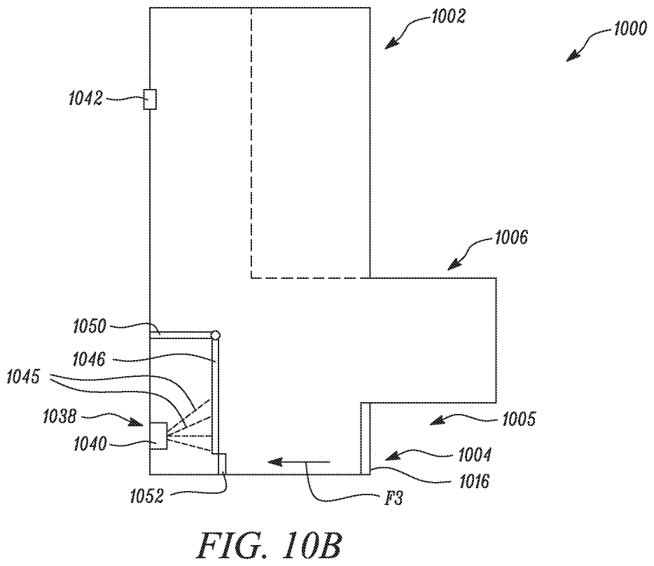

FIG. 10A illustrates another embodiment of an inhaler 1000. The inhaler 1000 is similar to the inhaler 600 explained in relation to FIG. 6. The inhaler 1000 includes an actuator housing 1002 and a mouthpiece 1006. In this embodiment, the air inlet 1004 is defined at a lower portion 1005 of the inhaler 1000. The air inlet 1004 includes an air inlet cover 1116 similar to the air inlet cover 116 depicted in FIG. 2. The inhaler 1000 includes a system 1038 that is similar to the system 200 described in relation to FIGS. 3 and 4. Further, the system 1038 includes the sensor 1040 and the feedback device 1042 that are similar in operation to the sensor 204 and the feedback device 206, respectively, explained in relation to FIGS. 3 and 4. The sensor 1040 is disposed generally parallel to an air flow path “F3” defined by the actuator housing 1002. In one example, a range of the sensor 1040 is programmed to be restricted to a predefined range above a periphery of the air inlet 1004 and/or the air inlet cover 1016 such that the sensor 1040 can only detect obstructions up to the predefined range above the periphery of the air inlet cover 1016.

When the sensor is activated, the sensor 1040 continuously transmits detection signals 1045. The sensor 1040 is programmed such that the air inlet cover 1016 constantly reflects the detection signals 1045 transmitted from the sensor 1040. Any obstruction of the air inlet 1004 alters the reflected signals that are detected by the sensor 1040. If the signal alteration caused by the obstruction exceeds a predefined signal change, an output signal is generated by the sensor 1040. The predefined signal change may be prestored in a memory of a control circuitry that may be associated with the sensor 1040 or the feedback device 1042.

Thus, if the object 202 comes within the range of the sensor 1040, the sensor 1040 detects the object 202 as the obstruction of the air inlet 1004 and generates the output signal. The feedback device 1042 receives the output signal from the sensor 1040 and generates a feedback signal indicative of the obstruction of the air inlet 1004. The feedback device 1042 provides the feedback signal to the patient regarding the obstruction of the air inlet 1004 based on the receipt of the output signal. For example, the feedback device 1042 may provide the feedback signal to the patient regarding the obstruction of the air inlet 1004 using an LED which may display or flash a color signature to notify the patient regarding the obstruction of the air inlet 1004, a buzzer sound that emits a sound frequency or tones to alert the patient, a haptic feedback, a text notification that may be displayed on a display device (not depicted) of the inhaler 1000, etc.

The inhaler 1000 may also include a cover member 1046. The cover member 1046 is movable between an open position and a closed position. The cover member 1046 is depicted in the open position in FIG. 10A and in the closed position in FIG. 10B. In one example, the cover member 1046 moves between the open and closed positions based on control signals received from the control module associated with the inhaler 100. More particularly, an actuator (not depicted) may move the cover member 1046 based on the control signals received from the control module. Further, a spring (not depicted) biases the cover member 1046 to the open position, such that the actuator moves the cover member 1046 against a biasing of the spring. The cover member 1046 is pivotally coupled to a first member 1050 such that the cover member 1046 pivots with respect to the first member 1050 to switch between the open and closed positions.

As depicted in FIG. 10B, the cover member 1046 is configured to cover the sensor 1040 in the closed position. More particularly, during the delivery of the medicament to the patient, the cover member 1046 covers the sensors 1040. In the closed position the cover member 1046 is in contact with a second member 1052. The cover member 1046 may include a flap, a vane, or a valve. The cover member 1046 moves between the open and closed positions to isolate the sensor 1040 from the air flow path “F3” during the delivery of the medicament to the patient.

In another example, the sensor 1040 may be further protected by a sensor shield such that the sensor shield does not affect a performance of the sensor 1040.

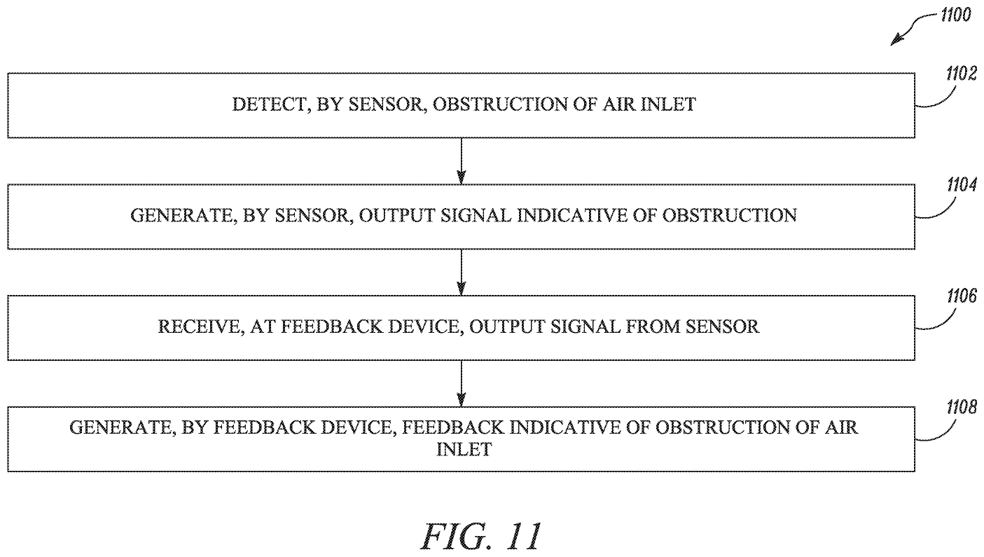

FIG. 11 illustrates a flowchart for a method 1100 of detecting the obstruction of the air inlet 104, 704, 904, 1004 of the inhaler 100, 700, 900, 1000 used for delivering the medicament to the patient. At step 1102, the sensor 204, 740, 940, 1040 detects the obstruction of the air inlet 104, 704, 904, 1004. The sensor 204, 740, 940, 1040 emits the detection signals 208, 745, 945, 1045 towards the air inlet 104, 704, 904, 1004 for detecting the obstruction of the air inlet 104, 704, 904, 1004. The sensor 204, 740, 940, 1040 is activated based upon the detection of the predefined inhaler preparation signature. Further, during the delivery of the medicament to the patient, the cover member 946, 1046 covers the respective sensors 940, 1040.

At step 1104, the sensor 204, 740, 940, 1040 generates the output signal indicative of the obstruction. At step 1106, the feedback device 206, 742, 942, 1042 receives the output signal from the sensor 204, 740, 940, 1040. At step 1108, the feedback device 206, 742, 942, 1042 generates the feedback signal. The feedback signal is indicative of the obstruction of the air inlet 104, 704, 904, 1004. The feedback signal includes at least one of an optical feedback signal, an audio feedback signal, and the haptic feedback signal.

It should be noted that positioning of the sensors 204, 740, 940, 1040 mentioned above may vary depending on a position of the respective air inlets 104, 704, 904, 1004 and the particular airflow path of an inhaler. The teachings of the present disclosure can be integrated into an inhaler or provided as an add-on device that can be attached to an inhaler. The system 200, 738, 938, 1038 can be applied to inhalers of the press-and-breathe type, breath-actuated type, dry powder type, soft mist type, etc., without limiting the scope of the present disclosure.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments depicted and described without departing from the scope of the present disclosure. This application is intended to cover any adaptations or variations of the specific embodiments discussed herein. Therefore, it is intended that this disclosure be limited only by the claims and the equivalents thereof.

1. An inhaler for delivering a medicament, the inhaler comprising:
an actuator housing for holding the medicament, the actuator housing comprising an air inlet for receiving air flow, the actuator housing further defining an air flow path into which the medicament is dispensed;
a sensor configured to detect an obstruction of the air inlet and to generate an output signal indicative of the obstruction; and
a feedback device configured to receive the output signal from the sensor and to generate a feedback signal.

2. The inhaler of claim 1, wherein the sensor is disposed on the actuator housing.

3. The inhaler of claim 1, wherein the actuator housing further comprises an air inlet cover proximate the air inlet, and wherein the sensor is mounted on the air inlet cover.

4. The inhaler of claim 1, wherein the feedback device is disposed on the actuator housing.

5. The inhaler of claim 1, further comprising an add-on device detachably connected to the actuator housing, wherein the sensor is disposed on the add-on device.

6. The inhaler of claim 5, wherein the feedback device is disposed on the add-on device.

7. The inhaler of claim 1, wherein the sensor is disposed generally perpendicular to the air flow path.

8. The inhaler of claim 1, wherein the sensor is disposed obliquely to the air flow path.

9. The inhaler of claim 1, wherein the sensor is disposed generally parallel to the air flow path.

10. The inhaler of claim 1, wherein the feedback device comprises at least one of an optical component, an audio component, and a haptic component.

11. The inhaler of claim 1, wherein the sensor is at least one of an ultrasonic sensor, an optical sensor, a laser detector, a force sensor, and a temperature sensor.

12. The inhaler of claim 1, wherein the sensor is further configured to emit detection signals towards the air inlet.

13. The inhaler of claim 1, wherein the feedback signal is indicative of the obstruction of the air inlet.

14. A method of detecting an obstruction of an air inlet of an inhaler, the inhaler used for delivering a medicament, the method comprising:
detecting, by a sensor, the obstruction of the air inlet;
generating, by the sensor, an output signal indicative of the obstruction;
receiving, at a feedback device, the output signal from the sensor; and
generating, by the feedback device, a feedback signal.

15. The method of claim 14, further comprising activating the sensor upon detection of a predefined inhaler preparation signature.

16. The method of claim 14, further comprising emitting detection signals towards the air inlet by the sensor.

17. The method of claim 14, wherein the feedback signal comprises at least one of an optical feedback signal, an audio feedback signal, and a haptic feedback signal.

18. The method of claim 14, wherein the feedback signal is indicative of the obstruction of the air inlet.

19. An add-on device for an inhaler for delivering medicament, the inhaler comprising an actuator housing for holding the medicament, the actuator housing comprising an air inlet for receiving air flow, the actuator housing further defining an air flow path into which the medicament is dispensed, the add-on device comprising:
a sensor configured to be disposed proximal to the air inlet, the sensor configured to detect an obstruction of the air inlet and to generate an output signal indicative of the obstruction; and
a feedback device configured to receive the output signal from the sensor and to generate a feedback signal indicative of the obstruction of the air inlet, wherein the add-on device is configured to be detachably connected to the actuator housing.
